(19) United States
(12) Patent Application Publication
(10) Pub. No.: US 2021/0057064 A1
(43) Pub. Date: Feb. 25, 2021

(54) SYSTEMS AND METHODS FOR FEDERATED SEARCHING AND RETRIEVAL OF MEDICAL RECORDS ACROSS DISPARATE DATABASES

(71) Applicant: MEDICOM TECHNOLOGIES INC., RALEIGH, NC (US)

(72) Inventors: CHASE BALLARD, RALEIGH, NC (US); BRENT GOLDSTEIN, RALEIGH, NC (US); TOMMY LUO, RALEIGH, NC (US); CHRIS WOODLIEF, RALEIGH, NC (US); MICHAEL ROSENBERG, RALEIGH, NC (US)

(21) Appl. No.: 17/001,589

(22) Filed: Aug. 24, 2020

Related U.S. Application Data
(60) Provisional application No. 62/891,315, filed on Aug. 24, 2019.

(51) Int. Cl. G16H 10/60; G06Q 10/10; G06F 16/908; G06F 16/903

(52) U.S. Cl. CPC G16H 10/60; G06F 16/903; G06F 16/908; G06Q 10/1095

(57) ABSTRACT

The present invention relates generally to for an automated system that allows relevant medical records for a patient to be identified among, and transferred between, disparate medical providers, where once transferred, the medical records are automatically reconciled for the requesting medical provider. The present invention facilitates the preservation of the continuum of care, and allows medical records to automatically be obtained for patients ahead of scheduled appointments or procedures.

### CROSS-REFERENCE TO RELATED APPLICATION

This application claims the benefit of U.S. Provisional Patent Application No. 62/891,315 entitled “SYSTEM AND METHOD FOR FEDERATED SEARCHING AND RETRIEVAL OF PATIENT RECORDS ACROSS DISPARATE DATABASES” filed on Aug. 24, 2019, and which is commonly owned, the disclosure of which is incorporated herein by reference in its entirety.

### BACKGROUND

### Field of the Invention

The present invention relates, in general, to systems and methods for automated federated searching of medical records across disparate databases, and between disparate medical providers, where prior medical records related to a scheduled patient appointment can be retrieved and reconciled by a treating physician prior to the scheduled patient appointment.

### Description of Related Art

Timely access to medical records is critical to providing quality patient care. Medical records, such as prior medical imaging and interpretations, a medical history, laboratory results, medications, and the like, can be crucial to properly diagnosing a patient's medical condition, as well as determining a treatment plan. It is common for patients to seek treatment with different medical providers, and inherently, patients typically have medical records stored with different, unrelated medical providers and healthcare facilities.

In order for a medical provider to obtain a patient's external medical records, the medical provider typically has to manually call external medical providers, physically fill out a paper-based request and fax the request to external medical providers. This scenario assumes that the medical provider and/or patient knows which particular external medical provider has relevant medical records for the patient. In the event this information is not available, the medical provider must call multiple external medical providers in hopes of locating the relevant medical records for the patient.

If such relevant medical records are identified, the medical provider must also reconcile the medical records to ensure that the records belong to the correct patient, so that records from different patients are not inadvertently linked or associated. Such reconciliation can be a time consuming and manually intensive process, especially in light of the tremendous patient volume that most medical provider see on a constant basis.

In the field of radiology, access to all relevant prior imaging studies for a patient is essential so that a radiologist can compare and/or reference prior imaging against a patient's new imaging study in order to provide an accurate and timely interpretation. For example, in the case of mammography, comparison of a screening mammogram to prior screening mammograms typically provides improved detection yields.

Therefore, there is a need for an automated system that allows relevant medical records for a patient to be identified among, and transferred between, disparate medical providers, where once transferred, the medical records are automatically reconciled for the requesting medical provider. As such, the present invention ultimately facilitates the preservation of the continuum of care, and allows medical records to automatically be obtained for patients ahead of scheduled appointments or procedures.

### SUMMARY

In one embodiment, the present invention relates to a method for automated federating searching and retrieval of prior medical records from a remote database, comprising: receiving appointment information by a server; extracting, by the server, a patient identifier and a procedure identifier from the appointment information; transmitting, by the server, the patient identifier and the procedure identifier to a virtual instance of the server; identifying, by the virtual instance of the server, a medical record located on the remote database related to the patient identifier and the procedure identifier; and copying, by the virtual instance of the server, the medical record to a local database.

A method for automated federating searching and retrieval of prior imaging studies from a DICOM database, comprising: receiving a data feed from a patient scheduling system by a server; parsing the data feed by the server to identify appointment information; extracting from the appointment information, by the server, a patient identifier, a procedure identifier, and an appointment date; calculating a query date by the server based on the appointment date and a pre-determined time offset; generated, by the server, a query for prior imaging studies, wherein the query includes the patient identifier and the procedure identifier; relaying, by the server, the query for prior imaging studies to the DICOM database on the query date; and identifying, by the server, an imaging study located on the DICOM database related to the patient identifier and the procedure identifier.

In yet another embodiment, the present invention relates to a system for automated federated searching and retrieval of medical records across disparate databases, comprising: a server coupled to a first medical provider and a second medical provider; a virtual machine coupled to the first medical provider, wherein the virtual machine is an instance of the server; an electronic health record (EHR) system coupled to the first medical provider; a data exchange interface communicatively coupled between the EHR system the virtual machine, wherein the virtual machine is configured to receive a data feed from the EHR system, and wherein the virtual machine is further configured to extract appointment information from the data feed; and a database coupled to the second medical provider, wherein the virtual machine is configured to transmit a query to the database in order to identify a medical record related to the appointment information.

### BRIEF DESCRIPTION OF THE DRAWINGS

These and other embodiments of the disclosure will be discussed with reference to the following exemplary and non-limiting illustrations, in which like elements are numbered similarly, and where:

FIG. 1 is a network architecture diagram of a system for automated federated searching of medical records across disparate medical providers, according to an embodiment of the invention;

FIG. 2 is a flowchart illustrating the steps of automated federated searching of medical records across disparate medical providers, according to an embodiment of the invention;

FIG. 3 is a flowchart illustrating the steps of transferring prior medical records from an external medical provider to a requesting medical provider, according to an embodiment of the invention;

FIG. 4 is a flowchart illustrating the steps of configuring automated federated searching of medical records across disparate medical providers, according to an embodiment of the invention;

FIG. 5 is a block diagram illustrating virtual machines and disparate medical provider databases, according to an embodiment of the invention; and

FIG. 6 is a network architecture diagram of a system for automated federated searching of medical records across databases within an organization, according to an embodiment of the invention.

### DEFINITIONS

The following definitions are meant to aid in the description and understanding of the defined terms in the context of the present invention. The definitions are not meant to limit these terms to less than is described throughout this application. Such definitions are meant to encompass grammatical equivalents.

As used herein, the term “medical data” can refer to, for example, health and healthcare related data, patient data, electronic medical records, medical imaging studies, medical and diagnostic images, diagnostic reports, fitness and activity data, medical reports, patient medical history and demographics, encounter and visit histories, admit/discharge/transfer and patient tracking information, scheduling and referrals, orders and results (measurements, observations, impressions, reports), pharmacy and diet information, and the like.

As used herein, the term “medical reports” can refer to, for example, any patient data, patient charts, diagnostic notes, opinions, reads and reports, medical test and laboratory results, surgical history, family history, medications, medical allergies, social history, habits (such as, for example, drug, tobacco and alcohol use), immunization history, clinical information, growth and development history, medical encounters, physical examination observations, progress notes, and the like.

As used herein, the term “medical provider” can refer to, for example, medical clinics, hospitals, medical providers, health insurance providers, diagnostic sites, imaging sites, data customers, and the like.

As used herein, the term “patient scheduling system” can refer to, for example, an electronic health record (EHR) system, electronic medical record system (EMR), a hospital information system (HIS), a Computerized Physician Order Entry systems (CPOE), and Integrating the Healthcare Enterprise Order Placer (IHE OP), a PACS, a Radiology Information System (RIS), a medical practice management system, a patient scheduling system, a physician referral system, and the like.

As used herein, the term “data exchange interface” can refer to, for example, an interface, standard, specification and/or protocol that allows for disparate systems to share data, including, but not limited to, the Health Level Seven (HL7) standard, the Fast Healthcare Interoperability Resources (FHIR) standard, the HPRIM standard, the Continuity of Card Record (CCR) standard, the Continuity of Care Document (CCD) specification, the xDT data exchange format, the Arden syntax, an application programming interface (API), and direct access protocols for electronic health records systems, and the like.

As used herein, the term “machine learning” can refer to, for example, deep learning, reinforcement learning, neural network computing, artificial intelligence computing, fuzzy logic computing, and the like.

### DETAILED DESCRIPTION

It should be understood that aspects of the invention are described herein with reference to the figures, which show illustrative embodiments. The illustrative embodiments herein are not necessarily intended to show all embodiments in accordance with the invention, but rather are used to describe a few illustrative embodiments. Thus, aspects of the invention are not intended to be construed narrowly in view of the illustrative embodiments. In addition, although the invention is described with respect to its application for the transfer of medical information containing DICOM data applications, it is understood that the system could be implemented in any setting where the transfer of any type or form of medical or patient data may be useful.

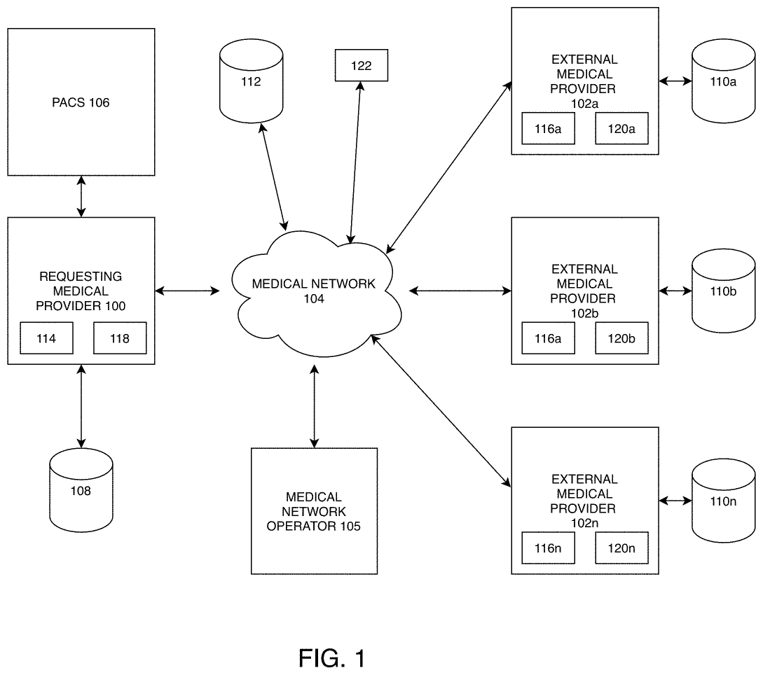

FIG. 1 is a network architecture diagram of a system for automated federated searching of medical records across disparate medical providers, according to an embodiment of the invention. In an embodiment, a requesting medical provider 100 is communicatively coupled to at least external medical provider 102 via a medical network 104. The medical network 104 can be a restricted network operated by a medical network operator 105, such that the medical providers 100, 102 are registered with the medical network 104, and have a relationship with the medical network operator 105.

In an embodiment, the medical network 104 includes computing hardware, software, and/or services that facilitate the automated federated searching of medical records associated with the external medical providers 102. In an embodiment, the medical network 104 is configured as a server, virtual server, or a distributed server that can store data, as well as execute programs, algorithms, scripts, and applications.

In an embodiment, the medical network 104 is a proprietary network that facilitates direct peer-to-peer data transfer between the medical providers 100, 102 as described in more detail in commonly owned application Ser. No. 16/281,409, entitled, METHODS AND SYSTEMS FOR TRANSFERRING SECURE DATA AND FACILITATING NEW CLIENT ACQUISITIONS, the contents of which are hereby incorporated by reference in its entirety.

In an embodiment, the requesting medical provider 100 is, or is coupled to, a medical facility, hospital, outpatient clinic, diagnostic imaging center, diagnostic imaging station, and the like. In an embodiment, the medical provider 100 is includes a picture archiving and communication system (PACS) 106. The requesting medical provider 100 can be remote from, or locally coupled to, the PACS 106, and the requesting medical provider 100 can be communicatively coupled to multiple local or distributed PACS (not shown).

In an embodiment, the requesting medical provider 100 include computing hardware and software, and can be coupled to a database 108 that contains medical records. Similarly, each external medical provider 102a-n can include computing hardware and software, and each can be coupled to a respective database 110a-n, as shown in FIG. 1. In an embodiment, the medical network operator 105 can be coupled to a database 112.

In an embodiment, the database 112 can be configured to store replicated versions of data stored on the databases 110a-n. The database 112 can be updated or refreshed to reflect real-time changes to each database 110a-n, or the database 112 can be updated or refreshed on a periodic basis, such as daily, weekly, monthly, etc. In an embodiment, the database 112 can be updated or refreshed based on a triggering event, such as for example, a request for medical records being received from a requesting medical provider 100 by the medical network 104.

Each database 108-112 can be locally stored and managed by its respective associated entity, and each database 108-112 can be distributed or cloud-based, and located remotely from its respective associated entity, such as on a remote server provided by Amazon Web Services® or the like. In an embodiment, each database 108-112 can be a relational database, a SQL database, an object-oriented database, a centralized database, or a distributed database, such as a cloud-based database or a blockchain-based database stored across a distributed ledger. In an embodiment, the databases 108 and 110 are capable of storing data and files in the DICOM format.

In an embodiment, any of the databases 108-112 can be remotely located from a requesting medical provider 102. In this context, a remote database can include a database that is located within a LAN, WAN, and/or intranet associated with the requesting medical provider 100, or the remote database can be located with an external medical provider 102, and outside of a LAN, WAN, and/or intranet associated with the requesting medical provider 100.

In an embodiment, each medical provider 100, 102 is configured to run a virtual machine 114, 116 that is operated, provided, and/or developed by the medical network operator 105, and are configured as a virtual instance of medical network server 122. In an embodiment, the virtual machine 114, 116 can include DICOM standard functionality, and is configured to interface with a patient scheduling system 118, such as, for example, an EHR system. The virtual machines 114, 116 are configured to interface with each respective patient scheduling system 118, 120 by, for example, a data exchange interface, such as the HL7 standard, the FHIR standard, the HPRIM standard, the CCR standard, the CCD specification, the xDT data exchange format, the Arden syntax, an API, direct access to an EHR and/or associated databases, and the like.

In an embodiment, the medical network 104 is communicatively coupled to each virtual machine 114, 16, and can receive messages, information, and the like from each virtual machine 114, 116. For example, the virtual machine 114 coupled to the requesting medical provider 100 can be configured to transmit a message to the medical network 104 when a patient appointment is entered into the patient scheduling system 118. In another example, the virtual machine 116 coupled to the external medical provider 102 can be configured to transmit a data refresh message to the medical network 104 when database 112 is updated or modified, indicating that the database 112 needs to refresh its data to reflect the updates to database 110.

In an embodiment, each virtual machine 114, 116 can function as an edge compute server which allows for distributed processing of data by physically being closer to each respective database 108, 110 and/or patient scheduling system 118, 120.

In another embodiment, the functions of each virtual machine 114, 116 can be executed by the medical network 104. In this embodiment, the medical network 104 includes a medical network server 122. In an embodiment, the medical network server 122 can be distributed across multiple computing resources within the medical network 104, or can be a dedicated computing resource.

In another embodiment, instead of a virtual machine, each medical provider 100, 102 can include a physical server that locally executes the functions described herein of the virtual machine.

In an embodiment, medical providers 100, 102 can be communicatively coupled to the medical network 104 via communication links (denoted by arrows in FIG. 1). These communication links may be any type of communication links suitable to allow interaction between the medical providers 100, 102, as well as with the medical network 104 and medical network operator 105. For example, the communication links may each be a wired network, a wireless network, or any combination thereof. Further, communication links may include a distributed computing network, an intranet, a local-area network (LAN) and/or a wide-area network (WAN), or any combination thereof. For example, the LAN may make use of WIFI in its many variations and the WAN may make use of broadband, cellular and/or satellite networks using technologies including, but not limited to, CDPD, CDMA, GSM, PDC, PHS, TDMA, FLEX, ReFLEX, iDEN, TETRA, DECT, DataTAC, Mobitex, EDGE and other 2G, 3G, 4G and LTE technologies. However, those of ordinary skill in the art will appreciate that the communication links are not limited thereto. In another embodiment, the communication links may each include ethernet, Firewire, parallel, serial, or USB connections, or short-range communication protocols such as Bluetooth, infrared, Zigbee, and the like.

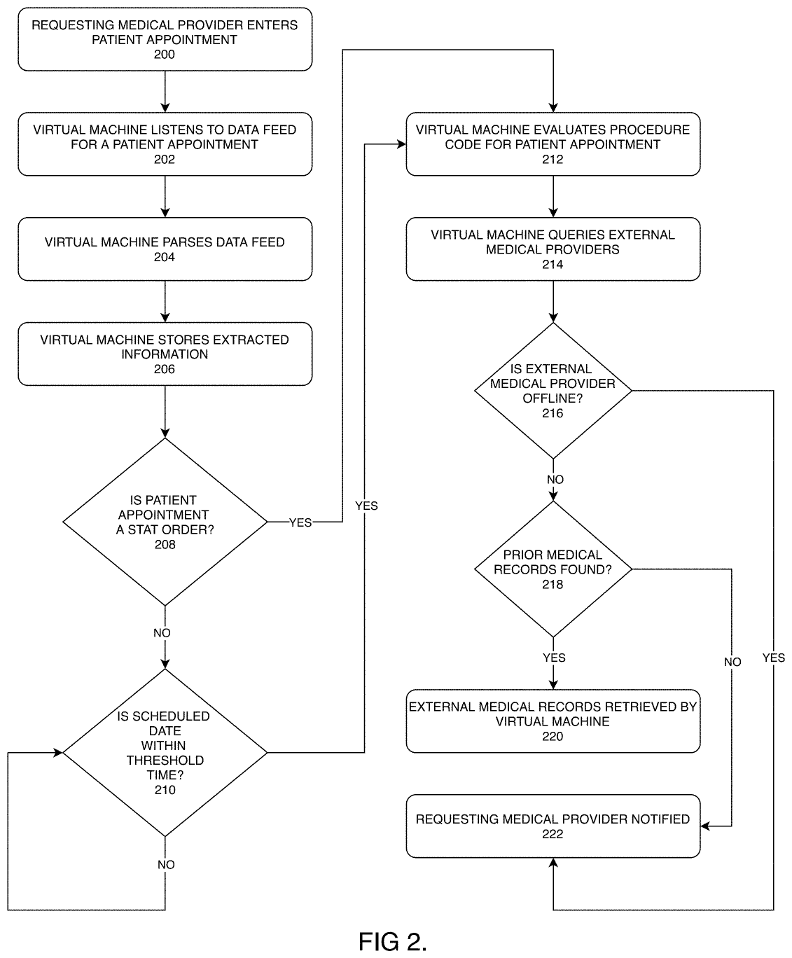

FIG. 2 is a flowchart illustrating the steps of automated federated searching of medical records across disparate medical providers, according to an embodiment of the invention. In step 200, the requesting medical provider 100 enters an appointment for a patient. The appointment can include, for example, appointment details, procedure identifiers and patient identifiers.

In an embodiment, the appointment details can include, but is not limited to an appointment date, appointment time, appointment facility/location, STAT or RUSH order instructions, etc., as well as prescribing physician and referred physician information, pre-appointment instructions, etc.

In an embodiment, the procedure identifiers can include, but are not limited to, procedure codes, CPT codes, LOINC codes, SNOMED codes, CPOE codes, procedure names, procedure codes, insurance billing codes, procedure name acronyms, procedure descriptions, modality types, body parts, organs, limbs and/or appendages related to the procedure, and the like.

In an embodiment, the patient identifiers can include, but are not limited to, a patient first, middle, and/or last name, date of birth, social security number, demographics, sex, address, telephone number, insurance information, primary care physician information, universal identifier, accession number, medical record number, and the like.

In an embodiment, the requesting medical provider 100 enters a patient appointment into a patient scheduling system 118 that is integrated with the EHR system via a portal (i.e., such as in a FHIR compatible EHR or in an EHR with API access) In an embodiment, the patient appointment can be generated remotely and electronically entered or pushed into the patient scheduling system 118, which can integrated into, or coupled with, for example, an EHR, a physician referral system, a laboratory testing ordering system, and the like.

In step 202, a message related to the patient appointment is transmitted to the virtual machine 114 by the patient scheduling system 118. In an embodiment, the message can include the appointment details, procedure identifiers, and patient identifiers. In an embodiment, the virtual machine 114 is configured to receive push notifications or messages from the patient scheduling system 118 in real-time. In another embodiment, the virtual machine 114 is configured to receive push notifications or messages from the patient scheduling system 118 periodically, such as based on a pre-defined interval of every minute, every thirty minutes, every hour, once daily, once weekly, and the like. The pre-defined interval can be selected by the requesting medical provider 100, and can be dynamically adjusted based on preference, patient volumes, medical facility type (i.e. critical care, emergency, and the like may require more frequent pushes versus a long-term care, recovery and rehabilitation facilities, and the like).

In another embodiment, in step 204, the virtual machine 114 listens to, and analyzes, a data feed from a patient scheduling system 118 to determine if a patient appointment has been entered into the patient scheduling system 118. In an embodiment, the virtual machine 114 is communicatively coupled to the patient scheduling system 118 via a data exchange interface. Such connectivity allows the virtual machine 114 to access, receive data from, or subscribe to, a data feed from the patient scheduling system 118. The virtual machine 114 can “listen” to the data feed, and analyze and monitor the data feed for various information. The data feed can provide various information to the virtual machine 114, including, but not limited to, patient appointments entered into, or received by, the patient scheduling system 118.

In an embodiment, the virtual machine 114 is configured to actively receive the data feed from the patient scheduling system 118 whenever a change, update, or access to the patient scheduling system 118 occurs. In another embodiment, the virtual machine 114 is configured to receive the data feed periodically, such as based on a pre-defined interval of every minute, every thirty minutes, every hour, once daily, once weekly, and the like. The pre-defined interval can be selected by the requesting medical provider 100, and can be dynamically adjusted based on preference, patient volumes, medical facility type (i.e. critical care, emergency, and the like may require more frequent data feed pushes versus a long-term care, recovery and rehabilitation facilities, and the like).

In another embodiment, the requesting medical provider 100 can manually request the data feed to be transmitted to the virtual machine 114. In another embodiment, information related to the patient appointment can be transmitted directly to the virtual machine 114, instead of the virtual machine 114 listening to the data feed and analyzing the data feed to identify a patient appointment.

Next in step 204, the virtual machine 114 parses the data feed to identify information related to the patient appointment, such as, for example, appointment details, procedure identifiers and patient identifiers. In an embodiment, the virtual machine 114 includes parsing engine to analyze text, characters, numbers, and alphanumeric contents located within the data feed. The parsing engine can be configured to accept as an input the data feed, and identify various medical and healthcare related lexicons, such as, for example, coding data, physician instructions, laboratory results, appointment information, and the like.

In an embodiment, the data feed may be in a HL7 format. The parsing engine may be configured to parse the data feed in the HL7 format, and may be configured to assign a dictionary to the data feed in the HL7 format, so that the data feed may be translated. The parser 116 may be configured to parse data that is formatted in a HPRIM, GMSIH, JAHIS format, or any other format suitable and compliant with Integrating the Healthcare Enterprise (IHE) standards. In addition, parsing engines dedicated to the different versions of HL7 as well as various other medical data standards and/or dedicated to other known formats may be utilized by the parsing engine without departing from the scope of the present invention. In an embodiment, the parsing engine can utilize XML parsing, text-based parsing, and the like.

In an embodiment, the virtual machine 114 may include one or more parsing engines, whereby a first parsing engine may be configured to parse any incoming information and/or distribute the incoming data to other parsing engines.

In an embodiment, the parsing engine can utilizes a machine learning function or algorithm to parse the data feed, as well as to contribute to a dynamically updated learning dictionary. By using machine learning analysis on information extracted from data feeds over time, the parsing engine can be optimized to predictively optimize text and character recognition and analysis functions.

In step 206, the virtual machine 114 extracts information from the data feed related to the patient appointment identified in step 204. The extracted information can include, for example, the appointment details, procedure identifiers and patient identifiers.

In an embodiment, the extracted information can further include a mapping location extracted from the data feed related to the patient appointment. The mapping location can include, but is not limited to, a DICOM location, a network location or space, a database location, and the like that is mapped to an attribute in the patient appointment.

The extracted information can be stored on database 112 coupled to the medical network 104, or can be stored on a local database coupled to the virtual machine 114.

In step 208, if the virtual machine 114 determines that the patient appointment contains STAT or RUSH order instructions, then the process continues to step 212. If, however, if the virtual machine 114 determines that the patient appointment does not contain STAT or RUSH order instructions, then the process continues to step 210.

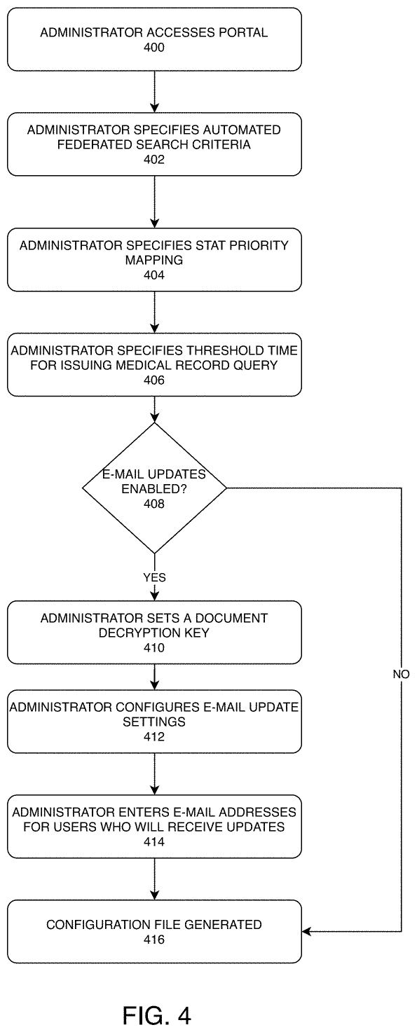

In step 210, the virtual machine 114 determines if the appointment date is within a time offset from the current date. In an embodiment, the time offset can pre-determined or pre-defined by the requesting medical provider 100, as described in more detail herein with reference to FIG. 4. In an embodiment, the virtual machine 114 calculates a query date based on the current date and the time offset (i.e., current date−time offset=query date).

For example, if the appointment date is 10 calendar days from the current date, and the time offset is 3 calendar days, then the virtual machine 114 does not take any action until 7 calendar days have passed (i.e., 10 calendar days−3 calendar days=7 calendar days).

If, however, the appointment date is 10 calendar days from the current date, and the time offset is 15 calendar days, then the virtual machine 114 proceeds with the automated federated searching process, as the query date criteria has been met (i.e., 10 calendar days−15 calendar days=−5 calendar days).

In an embodiment, the virtual machine 114 can utilize the appointment time (versus the appointment date), and compare it to a time offset based on a current time.

In an embodiment, the time offset can be in the form of calendar days, business days, a 24-hour time format, a 12-hour time format, and the like. In addition, the time offset calculation can compensate for existing holidays, scheduled closures, weekends, or known vacation of off-days for a referred physician.

In another embodiment, the time offset can be a delay. In this embodiment, the virtual machine 114 calculates a query day based on the current date and the time offset delay (i.e., current date+time offset=query date). For example, if the time offset is “2 weeks”, then the virtual machine 114 is configured to issue a prior medical record query two weeks after the patient appointment is generated. In another example, if the time offset delay is 3 calendar days, then the virtual machine 114 does not take any action until 3 calendar days have passed from the current date.

Next, in step 212, the virtual machine 114 analyzes the procedure information to determine a mapping of procedure codes to a modality type, such as a DICOM modality type. In an embodiment, the extracted CPT code(s) from the data feed can be analyzed and translated to specific DICOM modality types related to the patient appointment. For example, a CPT code of “74181” is for a “MM Abdomen w/o Contrast”, while a CPT code of “75572” is for a “CT Heart w Contrast”.

In an embodiment, the virtual machine 114 can generate a prior medical record query with the determined mapping, such that the prior medical record query is limited to prior medical data of the patient which may be relevant to the patient appointment. For example, if the patient appointment is for a “Mill Abdomen w/o Contrast”, the prior medical record query can specify, for example, “Mill” and “Abdomen” as a criteria. Thus, any prior medical records stored with an external medical provider 102 that is related to other modalities or body parts (i.e., an x-ray of a hand), would be deemed not relevant to the patient appointment, and such prior medical records would not be retrieved.

In an embodiment, the virtual machine 114 further analyzes extracted keywords and phrases from the data feed to determine body parts, organs, limbs and/or appendages which may be relevant to the patient appointment. For example, if the patient appointment includes text such as “left arm”, then the phrase “left arm” can be included as a criteria in the prior medical record query. In this example, any prior medical records related to a “right arm” would not be retrieved.

In step 214, the virtual machine 114 transmits a prior medical record query to the virtual machines 116a-n of the external medical providers 102. In an embodiment, the virtual machine 114 utilizes a C-FIND request which confirms to the DICOM standard. The C-FIND service is an operation by which relevant patient information and medical records can be queried across disparate databases.

The C-FIND request is transmitted to each virtual machine 116a-n of the external medical providers 100a-n from the virtual machine 114 of the requesting medical provider 100. The C-FIND request can be transmitted via the medical network 104. The C-FIND request can include various identifiers such as the appointment details, procedure identifiers, and patient identifiers, as well as the mapped procedure codes determined in step 212.

In another embodiment, the virtual machine 114 can query the virtual machines 116a-n using the WADO protocol, which can be utilized to obtain medical images stored on databases 110.

In step 216, the virtual machine 114 determines if an external medical provider 102 may be offline, or not connected to the medical network 104. In an embodiment, if a virtual machine 116 does not responds to the medical records request, or if an error is returned to the virtual machine 114, this may indicate that a particular external medical provider 100 may be offline.

If in step 216 the virtual machine 114 determines that an external medical provider 102 is offline, then the process continues to step 222, where the requesting medical provider 100 is notified as such. In an embodiment, the virtual machine 114 can generate a notification, alert, message, warning, and the like that is displayed in a portal accessible to the requesting medical provider 100. In an embodiment, the requesting medical provider 100 can receive an e-mail, secure message, text message, chat message, direct message, facsimile, electronic facsimile, physical printout, and the like.

In an embodiment, the virtual machine 114 determines if the database 110 is online, and not necessarily if the external medical provider 102 is online. In this embodiment, the virtual machine 114 determines if it can communicate or access the database 110, and if not, then the notification is generated. In an embodiment, the virtual machine 114 utilizes a C-ECHO request or a ping request to determine if the database 110 and functioning properly.

In an embodiment, the notification prompts the requesting medical provider 100 to manually intervene and contact the external medical provider 102 to request the medical records.

However, if in step 216 the virtual machine 114 determines that an external medical provider 102 is online, then the process continues to step 218, where the virtual machine 114 determines if the prior medical record query resulted in any matching prior medical records for the patient (i.e., prior medical records which relate to the procedure identifier). If the prior medical record query does not result in any matching prior medical records for the patient, then the process continues to step 222 where the requesting medical provider is notified as such. In an embodiment, the notification prompts the requesting medical provider 100 to manually intervene and contact the external medical provider 102 to verify that no matching prior medical records exist for the patient with the external medical provider 102.

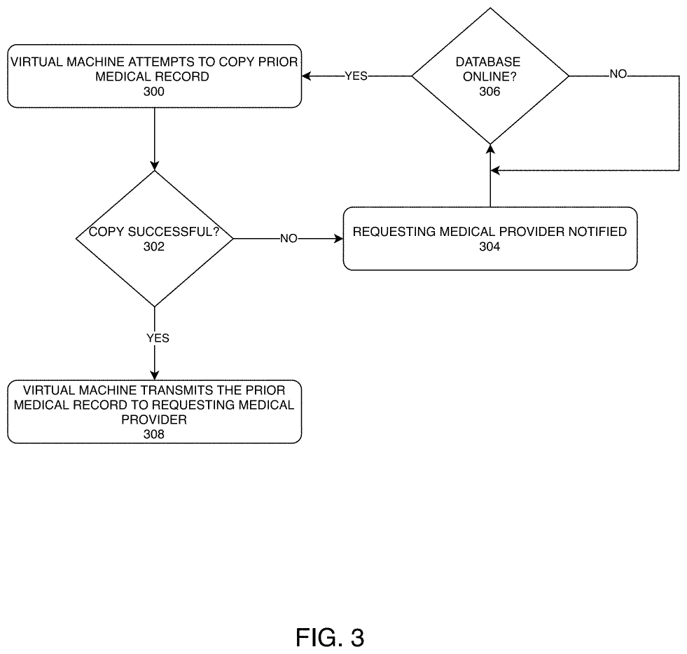

If the virtual machine 114 determines that matching prior medical records exist in the database 110, then the virtual machine 114 retrieves the prior medical records as described in more detail herein with reference to FIG. 3.

FIG. 3 is a flowchart illustrating the steps of transferring prior medical records from an external medical provider to a requesting medical provider, according to an embodiment of the invention. In step 300, the virtual machine 114 attempts to copy the prior medical record to a local database coupled to the virtual machine 114. In an embodiment, the virtual machine 114 can attempt to copy the prior medical record to database 112 coupled to the medical network 105.

Next, in step 302, the virtual machine 114 determines if it successfully copied the prior medical record. This determination can be made by a binary query to determine if the prior medical record exists in the local database of the virtual machine 114. In an embodiment, the virtual machine 114 can further compare the file size, header information, metadata, and the like of the copied version of the prior medical record to the original prior medical record to determine if the entire record was successfully copied.

In an embodiment, the virtual machine 114 utilizes a C-MOVE request which copies composite instances of the prior medical record to the virtual machine 114.

If the prior medical record was not successfully copied, then in step 304, the requesting medical provider 100 is notified as such. In an embodiment, the notification prompts the requesting medical provider 100 to manually intervene and contact the external medical provider 102 to request the prior medical record. The process then continues to step 306, where the virtual machine 114 determines if the database 110 is online, as described herein with reference to step 216 of FIG. 2. In an embodiment, the virtual machine 114 can persistently check if the database 110 is online. If the virtual machine 114 determines that the database 110 is online, the process continues to step 300, where the virtual machine 114 again attempts to copy the prior medical record to the local database of the virtual machine 114.

If however, the prior medical record is deemed to have been successfully copied in step 302, then the process continues to step 308, where the virtual machine 114 transmits the prior medical record to the requesting medical provider 308. In an embodiment, the virtual machine 114 is configured to transmit the prior medical record to the mapping location extracted in step 206 of FIG. 2. In an embodiment, based on the mapping location, the prior medical record can transferred via the medical network 105 to PACS 106, database 108, and/or patient scheduling system 118 of the requesting medical provider 100. In an embodiment, the virtual machine 114 can transmit the prior medical record to multiple mapping locations, or multiple destinations.

In an embodiment, the virtual machine 114 can reconcile the patient information in the prior medical record with the patient's information residing with the requesting medical provider 100. For example, the prior medical record may include a medical record number (MRN) for the patient associated with the external medical provider 102. The virtual machine 114 is configured to update the MRN to be consistent with the MRN of the patient associated with the requesting medical provider 100. In addition, any inconsistencies in the patient information that may be contained in the prior medical record can be corrected, or marked or flagged for manual human review. Such inconsistencies can include, but are not limited to, incorrect name spelling, date of birth, social security number, sex, address, telephone number, insurance information, primary care physician information, universal identifier, and the like.

In an embodiment, the virtual machine 114 can automatically correct any clear typographical errors, such as transposing of characters, apparent misspellings, etc. If the virtual machine 114 encounters inconsistencies such as differences which may be more significant than a typographical error, such as differences in a street address or insurance information, such inconsistencies can be marked or fluffed for manual human review.

In an embodiment, prior to transmitting the prior medical record to the mapping location, the virtual machine 114 can generate an unsolicited HL7 Observation Result (ORU) message or HL7 Order Entry (OE) message with an accession number. The accession number can be generated by the virtual machine 114 and/or the medical network 104. The accession number can be a unique identifier used by the medical network 104 to track, search, store, etc. prior medical records that have been accessed via the automated federated search process described herein, and the accession number is not necessarily linked to any local identification scheme utilized by any medical providers 100, 102.

The accession number can be used as part of a master patient index (MPI) managed by the medical network operator 105, which can identify patients and corresponding medical records across disparate medical providers, as well as separate clinical, financial, and administrative systems.

In an embodiment, if the mapping location is a DICOM location, then the requesting medical provider 100 and the external medical provider 102 from which the prior medical record originates must reside on the same network, such as intranet, local area network, or mesh network.

In another embodiment, if the requesting medical provider 100 and the external medical provider 102 reside on different networks, then the requesting medical provider 100 is required to enter a password or access code in order to complete the transfer of, or view, the prior medical record. In an embodiment, the requesting medical provider 100 can obtain the password from external medical provider 100, the patient, and/or the medical network operator 105.

FIG. 4 is a flowchart illustrating the steps of configuring automated federated searching of medical records across disparate medical providers, according to an embodiment of the invention. In step 400, an administrator can access a portal, such as a secure website via a Uniform Resource Location (URL) using a browser on a computing device. In an embodiment, the computing device can be a PACS station, a medical workstation, and the like.

The portal can be stored on, or executed from, for example, the medical network 104, and the portal allows the administrator to configure various settings and criteria for automated federated searching of medical records.

In an embodiment, prior to being able to access the portal, the administrator must enter credentials, such as a login and password, or other indicia that verifies their identity. The credentials can include user's mobile device number, login, password, e-mail address, phone number, account number, personal identification number (PIN), name, driver's license number, social security number, birthdate, employee number, and/or a unique account identification code previously provided to the administrator by an authorizing entity, such as an employer or the medical network operator 105. In another embodiment, the credentials can be biometric, such as a fingerprint, iris, facial, or voice scan. In yet another embodiment, the credential can be a gesture input by the user, such as a on a touchscreen or touchpad.

In an embodiment, the administrator is affiliated with the requesting medical provider 100, and can be an informational technology professional, medical record keeping personnel, medical office or practice manager, PACS administrator, a physician, a nurse, and the like.

In an embodiment, the administrator can be affiliated with the medical network operator 105. In this embodiment, the requesting medical provider 100 and medical network operator 105 can have a business arrangement or contractual agreement in place that allows the administrator to access the portal on behalf of the requesting medical provider 100. In this embodiment, the administrator may remotely access the portal via the virtual machine 114.

In step 402, the administrator specifies various automated federated search criteria, such as appointment details, procedure identifiers and patient identifiers. In an embodiment, the appointment details can include, but are not limited to an appointment date, appointment time, appointment facility/location, STAT or RUSH order instructions, etc., as well as prescribing physician and referred physician information, pre-appointment instructions, etc.

In an embodiment, the procedure identifiers can include, but are not limited to, CPT codes, LOINC codes, SNOMED codes, CPOE codes, procedure names, procedure codes, insurance billing codes, procedure name acronyms, procedure description, modality types, body parts, organs, limbs and/or appendages related to the procedure, and the like.

In an embodiment, the patient identifiers can include, but are not limited to, a patient first, middle, and/or last name, date of birth, social security number, demographics, sex, address, telephone number, insurance information, primary care physician information, universal identifier, accession number, medical record number, and the like.

For example, the administrator can specific if a middle name should be used as part of a prior medical record query. The inclusion of a patient's middle name is estimated to increase unique matches in a medical record data set from 95.7% to 98.9%.

In step 404, the administrator specifies a STAT or RUSH priority mapping. In an embodiment, the administrator can enter priority text strings, such as “stat”, “rush”, “asap”, “emergent”, “statum”, “immediate”, “no delay”, “critical”, “urgent”, “emergency”, and the like. If the virtual machine 114 identifies a priority text string in the data feed, then that particular patient appointment is classified as a STAT order, and the virtual machine 114 proceeds to generate and issue a prior medical record query as described herein.

If however the virtual machine 114 does not detect a priority text string in the data feed, then that particular patient appointment is classified as having a routine priority (i.e., not a STAT order), and the virtual machine 114 utilizes the time offset as described herein to calculate a query date.

Next, in step 406, the administrator specifies a time offset for issuing a prior medical record query. In an embodiment, the time offset can be a number of calendar or business days prior to a scheduled appointment date. In another embodiment, the time offset can be a delay which delays the query date based on a current date. The time offset is used by the virtual machine 114 to determine when to issue the prior medical record query.

For example, if the appointment date is 10 calendar days from the current date, and the time offset is 3 calendar days, then the virtual machine 114 does not take any action until 7 calendar days have passed (i.e., 10 calendar days−3 calendar days=7 calendar days).

If, however, the appointment date is 10 calendar days from the current date, and the time offset is 15 calendar days, then the virtual machine 114 proceeds with the automated federated searching process, as the query date criteria has been met (i.e., 10 calendar days−15 calendar days=−5 calendar days).

In an embodiment, the virtual machine 114 can utilize the appointment time (versus the appointment date), and compare it to a time offset based on a current time.

In an embodiment, the time offset can be in the form of calendar days, business days, a 24-hour time format, a 12-hour time format, and the like. In addition, the time offset calculation can compensate for existing holidays, scheduled closures, weekends, or known vacation of off-days for a referred physician.

In another embodiment, the time offset can be a delay. In this embodiment, the virtual machine 114 calculates a query day based on the current date and the time offset delay (i.e., current date+time offset=query date). For example, if the time offset is “2 weeks”, then the virtual machine 114 is configured to issue a prior medical record query two weeks after the patient appointment is generated. In another example, if the time offset delay is 3 calendar days, then the virtual machine 114 does not take any action until 3 calendar days have passed from the current date.

In another embodiment, the time offset can be dynamically updated if the scheduled appointment date is updated. In this embodiment, the virtual machine 114 is configured to analyze the data feed to determine if a particular patient appointment has any updates. If an updated appointment date or time is identified, then the time offset can be adjusted accordingly by the virtual machine 114.

Next, in step 408, the administrator has an option to enable e-mail updates. The e-mail update option allows additional users, such as medical professionals, individuals, care takers, and the like, to receive an update regarding the automated federated search status and prior medical record query results. In an embodiment, the update is not limited to an e-mail format, and the update can be in the form of a secure message, text message, chat message, direct message, facsimile, electronic facsimile, physical printout, and the like.

In an embodiment, the update is provided via an update document, which is preferably in the form of a PDF document. In an embodiment, the update document can be delivered as an encapsulated document, a Microsoft® Word document, and Open document, an Apple® Mac page, a rich text document, a StarOffice® document, a text document, a Corel® WordPerfect document, a Google® document, and the like.

In an embodiment, the update document can be encrypted, such that through encryption, the contents of the document, such as Protected Health Information (PHI), are transformed into a series of letters, numbers, symbols, and/or combinations thereof that is not human-perceivable or readable to help secure its content from unauthorized viewing or disclosure. The encrypted update document remains unperceivable until decrypted. Decrypting the document transforms the update document contents back into readable or perceivable form.

Encryption of the update document can help aid the user in compliance with privacy and confidentiality laws, industry standards, patient confidentiality, and any other type of federal and state privacy/confidentiality laws and regulations, such as, but not limited to, the Health Insurance Portability and Accountability Act (HIPAA).

If e-mail updates are enabled by the administrator, then the process continues to step 410, where the administrator sets a decryption key for the update document. The decryption key can be manually entered by the administrator, or can be a randomly generated key by the medical network server 122 or virtual machine 114.

In an embodiment, a different decryption key can be generated for each additional user. In an embodiment, the decryption key can be generated using a hash operation that utilizes as an input an identifier related to an additional user.

Once generated, the decryption key can be transmitted to the additional users, and the additional users are then required to enter or provide the decryption key in order to access an update document.

In step 412, the administrator can configure various e-mail update settings. In an embodiment, the administrator can set a daily time to send an aggregate e-mail update to the additional users. In an embodiment, the administrator can specify a different time for sending an e-mail update for each additional user.

In an embodiment, the portal indicates certain days and/or times at which the virtual machine 114 may be offline for maintenance, downtime, or scheduled service. The administrator is not able to select these offline times to schedule sending of the e-mail updates to the additional users.

In the event that the virtual machine 114 is unexpectantly offline during a scheduled e-mail update sending time, then the virtual machine 114 is configured to send the scheduled e-mail updates immediately upon returning to an online status, or alternatively, is configured to wait until a next scheduled e-mail update sending time (i.e., a next day, etc.).

In addition, the administrator can specify, for each additional user, if a particular additional user should receive an aggregate e-mail update or an individual e-mail update. In an embodiment, the administrator can select to redact or include certain content from an update document for each additional user. For example, the administrator can select to include PHI in update documents sent to medical professionals, and the administrator can select to redact PHI in update documents sent to non-medical professionals.

In step 414, the administrator can enter the e-mail address for each additional user. In an embodiment, the portal can display a directory of e-mail addresses for individuals affiliated with the requesting medical provider 100, the medical network operator 105, and/or the external medical providers 102. In an embodiment, the administrator can only enter e-mail addresses that are on the same network as the requesting medical provider 100, such as e-mail addresses of employees of the requesting medical provider 100.

In an embodiment, after the administrator enters an e-mail address, the additional user receives a confirmation e-mail which requires that the additional user to opt-in to receiving the e-mail updates. The opting-in function mitigates the risk of e-mail updates being sent to unintended users due to errors in the e-mail address input or selected by the administrator.

In step 416, the virtual machine 114 generates a configuration file, and the configuration file is uploaded to the database 112 coupled to the medical network 104. In another embodiment, the configuration file is stored on a local database coupled to the virtual machine 114. The configuration file can be retrieved at a later time by the administrator (or another administrator) in order to duplicate or modify the automated federated search settings. In an embodiment, the configuration file is in a comma-separated values (CSV) format, a JSON format, an XML format, a text format, and the like.

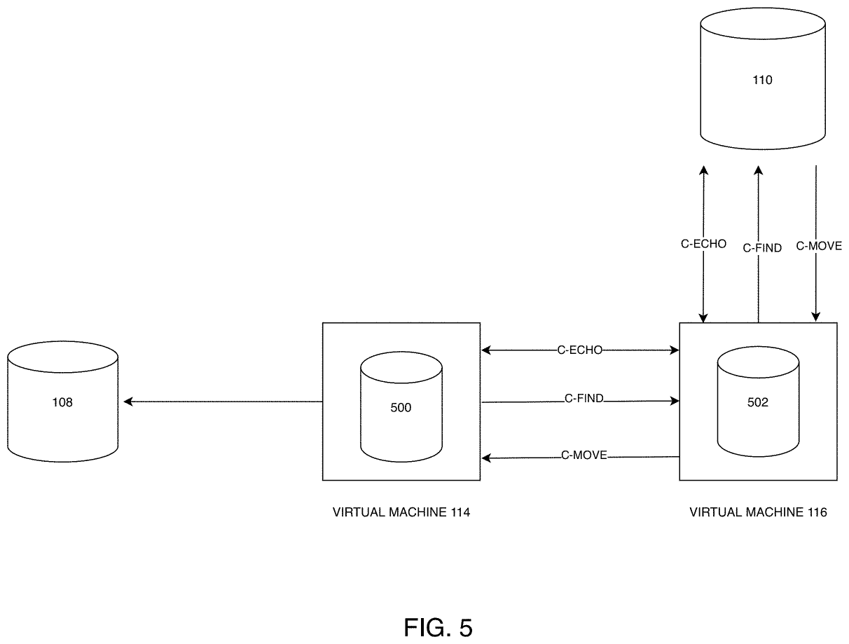

FIG. 5 is a block diagram illustrating virtual machines and disparate medical provider databases, according to an embodiment of the invention. In an embodiment, the virtual machine 114 includes a local database 500 coupled to the virtual machine 114. The local database 500 can reside within the virtual machine 114, or can be located remotely, such as integrated with database 112. In another embodiment, the database 112 is utilized instead of the local database 500.

In an embodiment, virtual machine 116 includes a similar or identical local database 502.

In an embodiment, the virtual machine 116 corresponding to an external medical provider 102 is coupled to a database 110 that contains medical records associated with the external medical provider 102. In order to issue a prior medical record query, the virtual machine 114 corresponding to the requesting medical provider 100 transmits a C-ECHO request to the virtual machine 116. In an embodiment, the virtual machine 116 relays the C-ECHO request to database 110 to determine if database 110 is online.

If the database 110 is online, the virtual machine 114 transmits a C-FIND request to the virtual machine 116 as described herein with reference to FIG. 2. The virtual machine 116 relays the C-FIND request to the database 110 to determine if any matching prior medical records exist on the database 110.

If matching prior medical records are identified, then the virtual machine 114 transmits a C-MOVE request to the virtual machine 116 as described herein with reference to FIG. 3. The virtual machine 116 relays the C-MOVE request to the database 110, and proceeds with the steps of copying the matching prior medical records as described herein with reference to FIG. 3.

In an embodiment, the matching prior medical records can be copied to local database 502 on virtual machine 116, or can be passed through directly to the local database 500 on virtual machine 115. In another embodiment, the matching prior medical records can be copied to database 112 coupled to the medical network 104.

Once copied, the virtual machine 114 transfers the copied prior medical records to the mapping location as described herein with reference to FIG. 2. The mapping location can be a location on database 108, PACS 106, and/or the patient scheduling system 118.

In an embodiment, the virtual machine 114 can communicate directly with database 110, and does not need to interface with virtual machine 116. In another embodiment, the requesting medical provider 100 can communicate directly with virtual machine 116, and does not need to interface with virtual machine 114. In this embodiment, the functions of virtual machine 114 described herein can be performed by virtual machine 116.

In an embodiment, the functions of the virtual machine 114 can be executed by the medical network server 122.

It is understood that any of the external medical providers 102 can also function as a requesting medical provider, and the requesting medical provider 100 can function as an external medical provider.

In yet another embodiment, the patient scheduling systems 118, 120 can perform the functions of the virtual machine 114, 116, and can operate an instance of the medical network server 122. In this embodiment where the patient scheduling systems 118, 120 are EHRs, each EHR can directly communicate with another EHR (i.e., direct EHR-to-EHR communication), where prior medical records can be transferred directly between EHRs located at disparate medical providers.

In an embodiment, the virtual machine 114 can leverage a machine learning function or algorithm in order to analyze various data that is processed by the virtual machine 114 and/or the medical network 104. For example, information extracted from the data feed over time, such as patient appointments, appointment details, procedure identifiers, and patient identifiers, can be stored in the local database 500 or database 112 coupled to the medical network 104. Similarly, information regarding inconsistencies in patient information in prior medical records between a requesting medical provider 100 and external medical provider 102 can be stored in the local database 500 or database 112. Such information can be analyzed over time by the machine learning function to identify trends, patterns, and insights that can be leveraged by the virtual machine 114 to predictively optimize the processes described herein for automated federated searching.

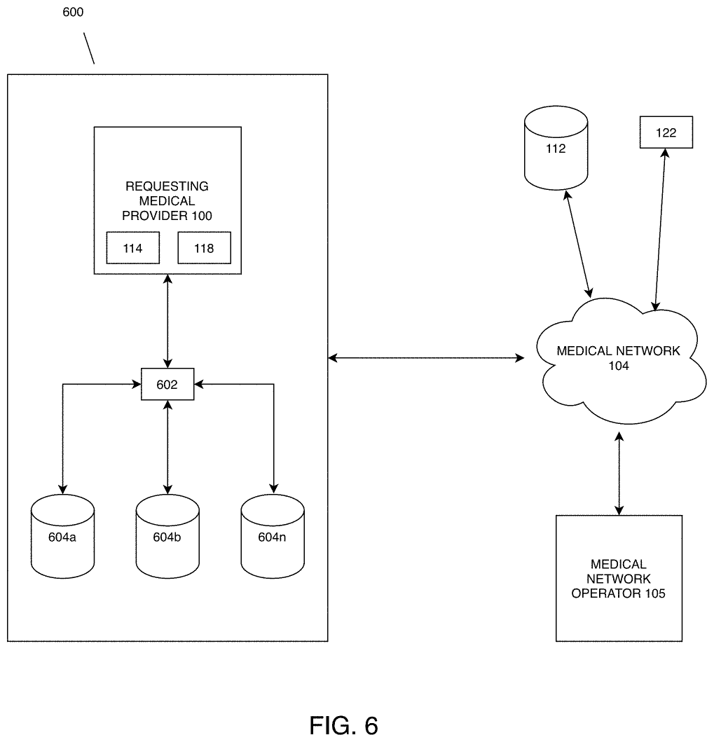

FIG. 6 is a network architecture diagram of a system for automated federated searching of medical records across databases within an organization, according to an embodiment of the invention. In an embodiment, a requesting medical provider 100 can have an internal communication network 600, such as a LAN, enterprise WAN, and/or intranet. In this embodiment, the requesting medical provider 100 can include a local server 602 that is communicatively coupled to multiple databases 604a-604n, and which is communicatively coupled to various components of the requesting medical provider 100, such as the virtual machine 114 and the patient scheduling system 118. In an embodiment, the functions of the virtual machine 114 can be executed by the medical network server 122.

In an embodiment, the requesting medical provider 100 can generate a prior medical record query via virtual machine 114, as described herein. Instead of transmitting the prior medical record query to an external medical provider 102, the virtual machine 114 can process the prior medical record query and can relay a C-FIND request to the databases 604a-604n within the internal communication network 600.

For example, mammography studies (and related imaging and reports) can oftentimes be stored on an internal or local database within an organization, such as a hospital having a hospital network coupled to various hospital computing devices and databases, such as for example, a PACS workstation and multiple PACS databases. The present invention allows a provider within the hospital network submit a prior medical record query within the hospital network and search the various local databases coupled to the hospital network. Thus, the local database can be a remote database in the sense that it is not necessarily coupled to a requesting provider's physical computing device, but the local database is located within the hospital network, such as a LAN, WAN, and/or intranet.

While the principles of the disclosure have been illustrated in relation to the exemplary embodiments shown herein, the principles of the disclosure are not limited thereto and include any modification, variation or permutation thereof.

What is claimed is:

1. A method for automated federating searching and retrieval of prior medical records from a remote database, comprising:
receiving appointment information by a server;
extracting, by the server, a patient identifier and a procedure identifier from the appointment information;
transmitting, by the server, the patient identifier and the procedure identifier to a virtual instance of the server;
identifying, by the virtual instance of the server, a medical record located on the remote database related to the patient identifier and the procedure identifier; and
copying, by the virtual instance of the server, the medical record to a local database.

2. The method of claim 1, further comprising extracting an appointment date from the appointment information by the server.

3. The method of claim 2, wherein the server transmits the patient identifier and the procedure identifier to the virtual instance of the server based on a time offset calculated using the appointment date.

4. The method of claim 1, wherein the procedure identifier is a CPT code.

5. The method of claim 1, wherein the procedure identifier is a body part.

6. The method of claim 1, wherein the patient identifier is a middle name.

7. The method of claim 1, wherein the server extracts the patient identifier and the procedure identifier from the appointment information using a parser.

8. A method for automated federating searching and retrieval of prior imaging studies from a DICOM database, comprising:
receiving a data feed from a patient scheduling system by a server;
parsing the data feed by the server to identify appointment information;
extracting from the appointment information, by the server, a patient identifier, a procedure identifier, and an appointment date;
calculating a query date by the server based on the appointment date and a pre-determined time offset;
generated, by the server, a query for prior imaging studies, wherein the query includes the patient identifier and the procedure identifier;
relaying, by the server, the query for prior imaging studies to the DICOM database on the query date; and
identifying, by the server, an imaging study located on the DICOM database related to the patient identifier and the procedure identifier.

9. The method of claim 8, further comprising extracting a mapping location from the appointment information by the server.

10. The method of claim 9, further comprising transferring, by the server, a copy of the imaging study to the mapping location.

11. The method of claim 8, wherein the procedure identifier is a body part.

12. The method of claim 8, wherein the database is a local database.

13. The method of claim 8, further comprising, monitoring the data feed by the server to determine if the appointment date has been modified.

14. A system for automated federated searching and retrieval of medical records across disparate databases, comprising:
a server coupled to a first medical provider and a second medical provider;
a virtual machine coupled to the first medical provider, wherein the virtual machine is an instance of the server;
an electronic health record (EHR) system coupled to the first medical provider;
a data exchange interface communicatively coupled between the EHR system the virtual machine, wherein the virtual machine is configured to receive a data feed from the EHR system, and wherein the virtual machine is further configured to extract appointment information from the data feed; and
a database coupled to the second medical provider,
wherein the virtual machine is configured to transmit a query to the database in order to identify a medical record related to the appointment information.

15. The system of claim 14, further comprising a second virtual machine coupled to the second medical provider.

16. The system of claim 14, wherein the data exchange interface utilizes a Health Level Seven (HL7) standard.

17. The system of claim 14, wherein the database is a DICOM database.

18. The system of claim 14, wherein the virtual machine is further configured to copy the medical record to a local database coupled to the first medical provider.

19. The system of claim 14, wherein the virtual machine is further configured to reconcile patient information contained in the medical record to be consistent with patient information associated with the first medical provider.

20. The system of claim 14, wherein the virtual machine is further configured to append an accession number to the medical record, and wherein the server utilizes the accession number in a master patient index.
